(19) United States
(12) Patent Application Publication
(10) Pub. No.: US 2018/0256278 A1
(43) Pub. Date: Sep. 13, 2018

(54) SMART PRESSURE-SENSITIVE MEASURING DEVICE FOR DISTAL FEMORAL TUMOR REMOVAL

(71) Applicant: TAT WAI YAU, HONG KONG (HK)

(72) Inventor: TAT WAI YAU, HONG KONG (HK)

(21) Appl. No.: 15/496,202

(22) Filed: Apr. 25, 2017

(30) Foreign Application Priority Data
Mar. 9, 2017 (CN) 201710138859.2

(51) Int. Cl. A61B 90/00; A61B 17/16

(52) U.S. Cl. CPC A61B 90/06; A61B 17/1664; A61B 2562/227; A61B 2090/067; A61B 2090/372; A61B 2090/061

(57) ABSTRACT

The present invention relates to a smart pressure-sensitive measuring device for distal femoral tumor removal comprising a rectangularly structured alignment plate, a measuring ruler and a measuring angle adjustment mechanism. An inverted T-shaped rail and a slide rail are disposed at the alignment plate. A magnetic plate is mounted at a bottom of the inverted T-shaped rail. The measuring angle adjustment mechanism comprises a T-shaped slider, a first locking mechanism, a readings disc and a second locking mechanism. The measuring ruler comprises a measuring ruler body, a pressure-sensitive display, a display control circuit board and a lithium battery. A cavity is disposed at one end of the measuring ruler body, the display control circuit board and the lithium battery is mounted inside the cavity. The measuring device has the advantages of being reasonable, simple, convenient, high accuracy, and high level of automation, can effectively solve the problem of traditional distal femoral tumor removal having low accuracy for measuring the length of distal end of femur.

### FIELD OF INVENTION

The present invention relates to the field of medical equipment, in particular to a smart pressure-sensitive measuring device for distal femoral tumor removal.

### BACKGROUND OF INVENTION

Existing distal femoral tumor removal surgery, or known as lower femur removal or lower femoral tumor removal, is a common surgery for treatment of sudden growth of chondroma, the tumor pressuring the nearby nerves and blood vessels and showing symptoms or affecting the function. In the operation of distal femoral tumor removal, the length of the distal end of femur needs to be measured, and a mark for osteotomy is made at a determined length before the surgery. Straight rulers are commonly used presently, using the knee joint surface as the initial point for measuring the length of the distal end of femur. Since the Q-angle of each patient can be different, the angle between the knee joint and anatomical axis of the distal end of the femur is a range, therefore it is difficult to use a measuring device having a fixed curvature for measurement. If straight ruler is used for measurement, the length of measurement is hard to fix, therefore also causes deviation, and affects surgery result.

### SUMMARY OF INVENTION

The technical problem to be solved by the present invention is: to overcome the aforementioned problem, a smart pressure-sensitive measuring device for distal femoral tumor removal is provided. The measuring device has the advantages of being reasonable, simple, convenient, high accuracy, and high level of automation, can effectively solve the problem of traditional distal femoral tumor removal having low accuracy for measuring the length of distal end of femur.

The technical solution used in the present invention to solve the technical problem is: a smart pressure-sensitive measuring device for distal femoral tumor removal comprising a rectangularly structured alignment plate, a measuring ruler and a measuring angle adjustment mechanism. An inverted T-shaped rail having a single opening is disposed on the alignment plate, a slide rail connected to the inverted T-shaped rail is disposed at a side of the alignment plate. A magnetic plate is mounted at a bottom of the inverted T-shaped rail.

The measuring angle adjustment mechanism comprises a T-shaped slider slidably connected to the inverted T-shaped rail, a first locking mechanism for locking the T-shaped slider, a readings disc and a second locking mechanism for locking the readings disc. A blocking plate, a screw shaft and a knob mounted on the blocking plate are disposed at the first locking mechanism the second locking mechanism respectively.

The screw shaft of the first locking mechanism is inserted from the slide rail, and is threaded to the T-shaped slider. The blocking plate is in contact with the outside wall of the alignment plate. A first reading for adjusting an angle between the measuring ruler and a right side wall of the alignment plate is disposed on the readings disc.

The measuring ruler comprises a measuring ruler body, a pressure-sensitive display mounted on the measuring ruler body, a display control circuit board and a lithium battery. A through slot is disposed at one end of the measuring ruler for adjusting angle. The screw shaft of the second locking mechanism is threaded to the T-shaped slider through the through slot and readings disc in such order, the blocking plate is further pressed against the measuring ruler body of the second locking mechanism. A cavity is disposed at another end of the measuring ruler body, the display control circuit board and the lithium battery is mounted inside the cavity, the display control circuit board is further electrically connected to the pressure-sensitive display and the lithium battery.

In a preferred embodiment, a cover plate is mounted on the opening of the inverted T-shaped rail.

In a preferred embodiment, the readings disc and the T-shaped slider is fixedly connected to each other.

In a preferred embodiment, the pressed location is set as 0-reading at the pressure-sensitive display, and displays a second reading that uses the 0-reading as the base and increases towards two sides in scale.

In a preferred embodiment, the second reading is two rows of readings symmetrically disposed relative to the center axis of the measuring ruler.

In a preferred embodiment, a USB charging port electrically connected to the display control circuit board is disposed on the measuring ruler body, a silicone plug is disposed on the USB charging port for preventing water and dust.

The advantage of the present invention is: smart pressure-sensitive measuring device for distal femoral tumor removal of the present invention comprises an alignment plate, a measuring ruler and a measuring angle adjustment mechanism. The measuring ruler and the alignment plate is connected to each other through the measuring angle adjustment mechanism, can adjust the angle depending on the circumstances of each patient. In use, the right side wall of the alignment plate is pushed towards the two circular contact surface of the femoral medial condyle and lateral condyle, the measuring ruler can slide freely inside the inverted T-shaped rail to adjust the position of the measuring ruler, being convenient to use and prevent measurement deviation of the length of distal end of the femur caused by the Q-angle of the knee. The pressure-sensitive display disposed on the measuring ruler uses the pressed location as 0-reading, and displays the second reading that uses the 0-reading as the base and increases towards the two sides in scale, facilitating the marking for surgeries, increasing the level of automation of the device. The adapted battery is a rechargeable battery, therefore having a longer operating life. The magnetic plate at the bottom of the inverted T-shaped rail of the alignment plate produces damping to the slider, making accurate positioning of the measuring ruler easier. The first locking mechanism can prevent measurement deviation caused by sliding of the measuring ruler. The measuring device has the advantages of being reasonable, simple, convenient, high accuracy, and high level of automation, can effectively solve the problem of traditional distal femoral tumor removal having low accuracy for measuring the length of distal end of femur.

### BRIEF DESCRIPTION OF DRAWINGS

The present invention is further explained below with the accompanying drawings and embodiments.

FIG. 1 is an overall structural schematic diagram of a smart pressure-sensitive measuring device for distal femoral tumor removal according to the present invention.

FIG. 2 is a side cross-sectional schematic diagram of a smart pressure-sensitive measuring device for distal femoral tumor removal testing according to the present invention.

FIG. 3 is a structural schematic diagram of the first locking mechanism of a smart pressure-sensitive measuring device for distal femoral tumor removal according to the present invention.

Reference numbers in the drawings: 1 alignment plate, 11 inverted T-shaped rail, 12 cover plate, 14 right side wall, 15 rail, 16 magnetic plate, 2 measuring ruler, 21 measuring ruler body, 22 pressure-sensitive display, 221 second reading, 23 through slot, 24 display control circuit board, 25 lithium battery, 26 cavity, 27 USB charging port, 3 measuring ruler angle adjustment mechanism, 31 first locking mechanism, 311 knob, 312 blocking plate, 313 screw shaft, 32 readings disc, 33 first reading, 34 second locking mechanism, 35 T-shaped slider.

### DETAILED DESCRIPTION OF INVENTION

The present invention is further explained below along with the accompanying drawings. The drawings are all simplified schematic diagrams which only illustrates the basic structure of the present invention, as such only the components material to the present invention are shown.

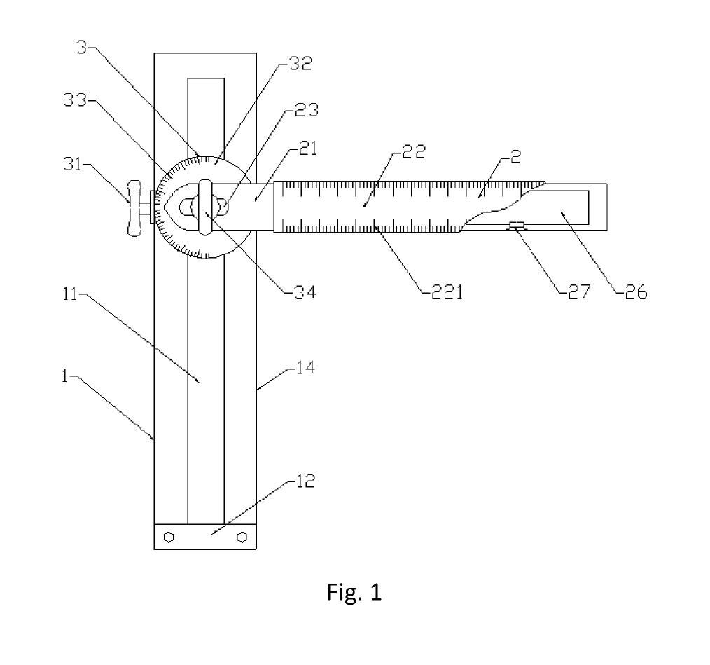

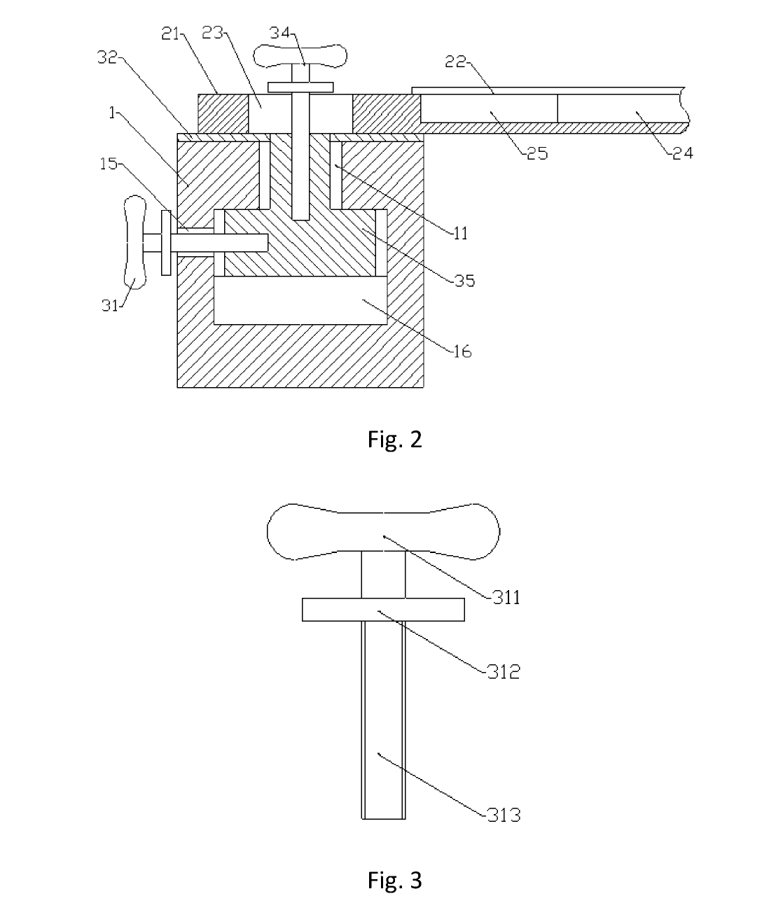

A smart pressure-sensitive measuring device for femoral tumor removal is shown in FIGS. 1-3. The device comprises a rectangularly structured alignment plate 1, a measuring ruler 2 and a measuring angle adjustment mechanism 3. An inverted T-shaped rail 11 having a single opening is disposed on the alignment plate, a slide rail 15 connected to the inverted T-shaped rail 11 is disposed at a side of the alignment plate 1. A magnetic plate 16 is mounted at a bottom of the inverted T-shaped rail 11.

The measuring angle adjustment mechanism 3 comprises a T-shaped slider 35 slidably connected to the inverted T-shaped rail 11, a first locking mechanism 31 for locking the T-shaped slider 35, a readings disc 32 and a second locking mechanism 34 for locking the readings disc 32. A blocking plate 312, a screw shaft 313 and a knob 311 mounted on the blocking plate 312 are disposed at the first locking mechanism 31 the second locking mechanism 32 respectively.

The screw shaft 313 of the first locking mechanism 31 is inserted from the slide rail 15, and is threaded to the T-shaped slider 35. The blocking plate 312 is in contact with the outside wall of the alignment plate 1. A first reading 33 for adjusting an angle between the measuring ruler 2 and a right side wall 14 of the alignment plate 1 is disposed on the readings disc 32.

The measuring ruler 2 comprises a measuring ruler body 21, a pressure-sensitive display 22 mounted on the measuring ruler body 21, a display control circuit board 24 and a lithium battery 25. A through slot 23 is disposed at one end of the measuring ruler 21 for adjusting angle. The screw shaft 313 of the second locking mechanism 34 is threaded to the T-shaped slider 35 through the through slot 23 and readings disc 32 in such order, the blocking plate 312 is further in contact with the measuring ruler body 21 of the second locking mechanism 34. A cavity 26 is disposed at another end of the measuring ruler body 21, the display control circuit board 24 and the lithium battery 25 is mounted inside the cavity 26, the display control circuit board 24 is further electrically connected to the pressure-sensitive display 22 and the lithium battery 25.

A cover plate 12 is mounted on the opening of the inverted T-shaped rail 11. The readings disc 32 and the T-shaped slider 35 is fixedly connected to each other. The pressed location is set as 0-reading at the pressure-sensitive display 22, and displays a second reading 21 that uses the 0-reading as the base and increases towards two sides in scale. The second reading 21 is two rows of readings symmetrically disposed relative to the center axis of the measuring ruler 2. A USB charging port 27 electrically connected to the display control circuit board 24 is disposed on the measuring ruler body 21, a silicone plug is disposed on the USB charging port 27 for preventing water and dust.

The smart pressure-sensitive measuring device for distal femoral tumor removal of the present invention comprises an alignment plate, a measuring ruler and a measuring angle adjustment mechanism. The measuring ruler and the alignment plate is connected to each other through the measuring angle adjustment mechanism, can adjust the angle depending on the circumstances of each patient. In use, the right side wall of the alignment plate is pushed towards the two circular contact surface of the femoral medial condyle and lateral condyle, the measuring ruler can slide freely inside the inverted T-shaped rail to adjust the position of the measuring ruler, being convenient to use and prevent measurement deviation of the length of distal end of the femur caused by the Q-angle of the knee. The pressure-sensitive display disposed on the measuring ruler uses the pressured location as 0-reading, and displays the second reading that uses the 0-reading as the base and increases towards the two sides in scale, facilitating the marking for surgeries, increasing the level of automation of the device. The adapted battery is a rechargeable battery, therefore having a longer operating life. The magnetic plate at the bottom of the inverted T-shaped rail of the alignment plate produces damping to the slider, making accurate positioning of the measuring ruler easier. The first locking mechanism can prevent measurement deviation caused by sliding of the measuring ruler. The measuring device has the advantages of being reasonable, simple, convenient, high accuracy, and high level of automation, can effectively solve the problem of traditional distal femoral tumor removal having low accuracy for measuring the length of distal end of femur.

As revealed by the preferred embodiments of the present invention, based on the aforementioned description, a person skilled in the art can make various modifications and adjustments without departing from the technical concept of the present invention. The technical scope of the present invention is not limited to the specification but is defined by the scope of the claims.

What is claimed is:

1. A smart pressure-sensitive measuring device for distal femoral tumor removal comprising: a rectangularly structured alignment plate, a measuring ruler and a measuring angle adjustment mechanism; an inverted T-shaped rail having a single opening is disposed on the alignment plate, a slide rail connected to the inverted T-shaped rail is disposed at a side of the alignment plate; a magnetic plate is mounted at a bottom of the inverted T-shaped rail;
the measuring angle adjustment mechanism comprises a T-shaped slider slidably connected to the inverted T-shaped rail, a first locking mechanism for locking the T-shaped slider, a readings disc and a second locking mechanism for locking the readings disc; a blocking plate, a screw shaft and a knob mounted on the blocking plate are disposed at the first locking mechanism the second locking mechanism respectively;
the screw shaft of the first locking mechanism is inserted from the slide rail, and is threaded to the T-shaped slider; the blocking plate is in contact with the outside wall of the alignment plate;
a first reading for adjusting an angle between the measuring ruler and a right side wall of the alignment plate is disposed on the readings disc;
the measuring ruler comprises a measuring ruler body, a pressure-sensitive display mounted on the measuring ruler body, a display control circuit board and a lithium battery; a through slot is disposed at one end of the measuring ruler for adjusting angle; the screw shaft of the second locking mechanism is threaded to the T-shaped slider through the through slot and readings disc in such order, the blocking plate is further pressed against the measuring ruler body of the second locking mechanism; a cavity is disposed at another end of the measuring ruler body, the display control circuit board and the lithium battery is mounted inside the cavity, the display control circuit board is further electrically connected to the pressure-sensitive display and the lithium battery.

2. The smart pressure-sensitive measuring device for distal femoral tumor removal according to claim 1, characterized in that: a cover plate is mounted on the opening of the inverted T-shaped rail.

3. The smart pressure-sensitive measuring device for distal femoral tumor removal according to claim 1, characterized in that: the readings disc and the T-shaped slider is fixedly connected to each other.

4. The smart pressure-sensitive measuring device for distal femoral tumor removal according to claim 1, characterized in that: the pressed location is set as 0-reading at the pressure-sensitive display, and displays a second reading that uses the 0-reading as the base and increases towards two sides in scale.

5. The smart pressure-sensitive measuring device for distal femoral tumor removal according to claim 4, characterized in that: the second reading is two rows of readings symmetrically disposed relative to the center axis of the measuring ruler.

6. The smart pressure-sensitive measuring device for distal femoral tumor removal according to claim 1, characterized in that: a USB charging port electrically connected to the display control circuit board is disposed on the measuring ruler body, a silicone plug is disposed on the USB charging port for preventing water and dust.
